(19) United States
(12) Patent Application Publication
(10) Pub. No.: US 2005/0065489 A1
(43) Pub. Date: Mar. 24, 2005

(54) Disposable Absorbent Artcle with Wetness Indicator

(76) Inventors: Stacy J. Driskell, Royersford, PA (US); Edward P. Erdman, West Chester, PA (US); Ruth L. Levy, Collegeville, PA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/668,599

(22) Filed: Sep. 24, 2003

(51) Int. Cl. A61F 13/15; A61F 13/20

(52) U.S. Cl. 604/361

(57) ABSTRACT

Absorbent articles that have wetness indicator graphics positioned thereon are disclosed, whereby the articles preferably include diapers, training pants, and adult incontinence products. The article includes a character graphic positioned in a waist region of the article, and a wetness indicator graphic that may be positioned in a crotch region. The character graphic is related to the wetness indicator graphic. The arrangement of the respective graphics preferably allow a caregiver to interact with a child to promote toilet training.

### BACKGROUND OF THE INVENTION

1. Field of The Invention

Embodiments of the invention relate to the field of absorbent articles in general, and more particularly to absorbent articles that include wetness indicators. Preferably, the disposable absorbent article, for example, a training pant, includes wetness indicators that are present (or not present) when the article is dry, and that disappear (or appear) when the article is wetted. The wetness indicators preferably allow a caregiver to interact with a child to promote toilet training.

2. Description of Related Art

Disposable absorbent articles typically include a moisture-impervious backing sheet, an absorbent pad, and a moisture permeable liner sheet (e.g., top sheet) that contacts the body of a person wearing the article. In addition, elasticized regions are provided around the edges of the article to secure the article about the waist and legs of a wearer. Diapers typically further comprise opposed front and rear waist portions defining a waist opening, a crotch portion disposed there between, and a pair of elastically contractible leg openings along the side edges of the crotch portion. Disposable diapers having elasticized margins for placement about the legs of a wearer are disclosed in U.S. Pat. No. 4,050,462 and U.S. Pat. No. 5,092,861. An absorbent article having elasticized side margins and waist band margins are shown in U.S. Pat. No. 4,300,562. These elasticized portions of the garment typically are designed to contain body exudates and hence, prevent leakage.

Training pants today typically are designed to include a number of different types of graphics in an attempt to train the child to go to the bathroom on the toilet, and to persuade a child to wear conventional undergarments. For example, some of these graphics include markings that make the training pants look like conventional boys or girls undergarments with the seams highlighted or emphasized to make the garment appear more like a conventional brief (e.g., simulated fly openings for boys, ruffles for girls, etc.).

Potty training a young child typically includes a wide variety of different aspects, including many training techniques and training aids that may be used by parents and caregivers, hereinafter simply referred to as caregivers. One feature of potty or toilet training is having the young child change from wearing diapers to wearing training pants to help the child understand that he or she should now use the toilet just like adults. An additional feature of the potty training process includes caregiver instruction as a positive encouragement and reinforcement to the child that he or she should now be using a toilet instead of diapers. Although the use of training pants and positive encouragement from the caregiver has been helpful in the toilet training process, there remains room for improvement. Specifically, caregivers continue to search for easier and quicker ways to guide their children successfully through the potty training process.

Many caregivers and parents have difficulty in determining when the child is ready to begin potty training, and underestimate the difficulty of teaching young children to use the toilet like an adult. Parents attempt many different forms of positive enforcement, from potty stickers or stamps, to using floating objects in the toilet. If a child does not respond to an initial toilet training instruction or introduction, the caregiver may be at a loss for finding techniques, methods, or teaching tools to encourage the child to use the toilet. Thus, while various teaching tools such as books, videotapes, charts with stickers, personalized toilets, and interactive toilet training kits are available, there remains a need for new and improved educational and motivational mechanisms to facilitate the toilet training process.

Another problem facing caregivers is that they typically do not know when a child has had an accident in his or her training pants, which can help in the potty training process since the accident will still be fresh in the child's mind. Wetness indicators have been used as a mechanism to assist caregivers in knowing when the articles has been wetted. The art is rife with disclosures on various types of wetness indicators, whether they be disappearing inks, appearing inks, inks activated by heat and the like, etc. Typical wetness indicators are disclosed, for example, in U.S. Pat. Nos. 4,022,211, 4,292,916, 4,812,053, 4,903,254, 4,987,849, 5,045,283, 5,503,339, 5,058,088, 6,297,424, and 6,307,119, the disclosures of each of which is incorporated by reference herein in their entirety.

U.S. Pat. Nos. 6,297,424 and 6,307,119 disclose the use of wetness indicators (called “active object graphics”) and their relationship with other graphics on the training patent (e.g., being “interactively interrelated” or “unrelated in subject matter”). The relationship between the various graphics, as well as their positioning, or registration, on the absorbent article, is said to assist the caregiver in telling a story to the child to assist in the potty training process. The use of these complicated interrelationships and registered positioning adds significantly to the cost of training pants, however, making them an undesirable purchase for most caregivers.

FIG. 7 is excerpted from U.S. Pat. No. 6,297,424. This figure reveals a complicated design requiring specific registration of the character (the dog in the boat) and the wetness indicators (the fish). These graphics typically are printed on different components of the absorbent article, thus requiring specific timing during the garments manufacture to ensure that the characters adequately interact with the wetness indicators. Registration of the character graphic in the waist portion, and the wetness indicators in the crotch portion of the garment is disclosed in, for example, U.S. Pat. No. 5,766,389, and its PCT counterpart WO97/24283, the disclosures of which are incorporated herein by reference in their entirety. This registration process is costly and time consuming.

The description herein of the various known products, methods, and apparatus and their attendant disadvantages is in no way intended to limit the scope of the present invention, or to imply that the present invention does not include some or all of the various elements of the products, methods, and apparatus in one form or another. Indeed, various embodiments of the invention may be capable of overcoming some of the disadvantages noted herein, while still retaining some or all of the various elements of the known products, methods, and apparatus in one form or another.

All documents described herein are incorporated by reference in this disclosure in their entirety.

### SUMMARY OF THE INVENTION

It is desirable to provide cost-efficient absorbent articles that assist in the potty training process. It also is desirable to provide absorbent articles that assist a caregiver in teaching a young child to use the toilet using positive reinforcement.

In accordance with features of various embodiments of the present invention, there is provided an absorbent article having a waist portion positioned adjacent one longitudinal end, and a crotch portion adjacent the waist portion. The absorbent article includes at least one unregistered character graphic positioned in the waist portion, and at least one wetness indicator positioned in the crotch portion. The wetness indicator and character graphic are related to one another, but not interactively interrelated.

In accordance with an additional feature of an embodiment of the invention, there is provided an absorbent article that includes a top sheet, a back sheet, and an absorbent core positioned at least partially between the top sheet and the back sheet. The absorbent article includes at least one wetness indicator positioned on the absorbent core facing surface of the back sheet, and at least one unregistered character graphic positioned in a waist portion of the absorbent article. The wetness indicator and character graphic are related to one another, but not interactively interrelated.

In accordance with an additional feature of an embodiment of the invention, there is provided a method of making an absorbent article that includes providing a top sheet, a back sheet, and an absorbent core to a garment forming station. The method includes positioning the absorbent core at least partially between the top sheet and the back sheet, and then positioning at least one wetness indicator at least partially between the back sheet and the absorbent core. The method further includes positioning at least one unregistered graphic in a waist portion of the absorbent article. The wetness indicator and unregistered character graphic are related to one another, but not interactively interrelated.

In accordance with preferred embodiments of the method, the graphics can be positioned on the article at the same or different times. In addition, the wetness indicators can be printed directly on a surface of the back sheet, or on an additional layer(s) and then positioned between the absorbent core and the back sheet. Furthermore, the unregistered character graphic can be printed on an additional layer(s) and then positioned in the waist portion of the article.

The absorbent article may include any number of layers in addition to the back sheet, top sheet and absorbent core, and the wetness indicators may be imprinted or otherwise positioned on the same or different ones of these layers. The absorbent article also may include additional graphics that may or may not be related to the wetness indicators including, for example, other object graphics, graphics or art simulating seam lines to make the article appear more pant like, and the like.

An additional feature of an embodiment of the invention includes an absorbent article having a waist portion positioned adjacent one longitudinal end, and a crotch portion adjacent the waist portion. The absorbent article includes at least one character graphic positioned in the waist portion, and at least one wetness indicator positioned in the crotch portion. The wetness indicator is related to the character in the character graphic and not to what the character may or may not be doing.

These and other features of various embodiments of the invention will be readily apparent from a review of the detailed description of preferred embodiments taken in conjunction with the attached drawings.

### BRIEF DESCRIPTION OF THE DRAWINGS

FIG. 1 is a front plan view of a pants-type absorbent garment with portions of the back sheet removed to reveal inner components of the garment;

FIG. 2 is a schematic of the various parts of a pants-type absorbent garment;

FIG. 3 is a front view of a preferred embodiment of the invention showing the relationship between the wetness indicator and the character graphic;

FIG. 4a illustrates a web of material with a series of character graphics; and FIG. 4b illustrates a web of material with a series of wetness indicators;

FIGS. 5a and 5b are front views of preferred embodiments of the invention showing the relationships between the wetness indicator and the character graphic;

FIG. 6 is a schematic illustrating an embodiment of a method of making an absorbent article including the graphic elements; and

FIG. 7 is a front view of a prior art training pant having wetness indicators interactively interrelated to the character graphic.

### DETAILED DESCRIPTION OF PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

Absorbent garments and diapers may have a number of different constructions. In each of these constructions it is generally the case that an absorbent core is at least partially disposed between a liquid pervious, body-facing top sheet, and a liquid impervious, exterior back sheet. In some cases, one or both of the top sheet and back sheet may be shaped to form a pants-like garment. In other cases, the top sheet, back sheet and absorbent core may be formed as a discrete assembly that is placed on a main chassis layer and the chassis layer is shaped to form a pants-like garment. The garment may be provided to the consumer in the fully assembled pants-like shape, or may be partially pants-like and require the consumer to take the final steps necessary to form the final pant-like shape. In the case of training pants-type garments for children and adult incontinent pants-type garments for adults, the garment is provided fully formed with factory-made side seams and the garment is donned by pulling it up the wearer's legs. In the case of diapers, which sometimes are used to train a child to go the bathroom, a caregiver usually wraps the diaper around the wearer's waist and joins the side seams manually by attaching one or more adhesive or mechanical tabs, thereby forming a pant-like structure.

For clarity, the present invention is described herein only with reference to a pants-type garment in which the top sheet, back sheet and absorbent core are assembled onto a chassis layer that forms a pants-like garment, although the invention may be used with other constructions such as diapers and adult incontinent products, and it is readily apparent and understood that this is not intended to limit the invention. The present invention may be used with any other absorbent garment that can be used as a teaching aid to teach a wearer (e.g., young child, accident victim, elderly person, etc.) to use the toilet. In addition, the invention can be used with an adult product whereby the graphic elements provide a mechanism to reveal in an aesthetically pleasing and dignified manner when the incontinent product has been wetted.

The term “component” can refer, but is not limited to designated selected regions, such as edges, corners, sides or the like; structural members, such as elastic strips, absorbent pads, stretchable layers or panels, layers of material, or the like; or a graphic.

Throughout this description, the term “positioned” and the expressions “positioned on,” “positioning on,” “positioned in,” “positioned between” and variations thereof (e.g., a description of the article being “positioned” is interposed between the words “positioned” and “on”) are intended to mean that one element can be integral with another element, or that one element can be a separate structure bonded to or placed with or placed near another element. Thus, a component that is “positioned on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent composite core. It is understood that additional layers may be present between the absorbent composite core and the top sheet and back sheet, and that additional layers and other materials may be present on the side opposite the absorbent composite core from either the top sheet or the back sheet. The expression “back sheet” as used herein, denotes the fluid impermeable material that prevents or impedes fluid contained in the absorbent core from escaping. A preferred embodiment of the invention includes an additional material covering the back sheet, which is referred to herein as the “outer cover.”

Throughout this description, the expression “wetness indicator” or “wetness indicator graphic” denotes any graphical representation that disappears from view when the garment is wetted, or appears over a period of time or when wetted. The presence or absence of the wetness indicator (i.e., disappearance or appearance), typically occurs when the child has an accident and the wetness indicator graphic is contacted with urine, but also when the product is in use and the disappearance or appearance occurs over time as a result of exposure to the environment, such as the air. The design of the wetness indicator graphic is not critical to the invention so long as the wetness indicator is “related” to the character graphic. Preferably, the wetness indicator graphic is a character, a graphical representation, a line, a series of lines, or the like.

Throughout this description, the expression “character graphic” denotes a graphic containing an anthropomorphous image, and in particular an image having or suggesting human form or appearance which ascribes human motivations, characteristics or behavior to inanimate objects, animals, natural phenomena, cartoon characters, or the like. Ideally the character graphic would be suitable for children's underwear and could be utilized to motivate children to wear the pants and use a potty or toilet. To that end, the character graphics can be associated with popular characters in the media, advertising or well known in a particular culture. Ideally, the character graphics are characters that the child or caregiver care about and want to identify with. Alternatively, when the graphics are positioned on an adult product, the character graphic may be a graphic that adults are familiar with, or with which adults have an association.

The character graphic can desirably comprise a portion of the entire training pant graphic that sets up a theme for the illustrated scene. As such, the character graphic can provide an opportunity for educational interaction between the child and the parent or caregiver. More specifically, the parent or caregiver can use the graphic story-line to make up a game or story for the purpose of toilet training progress.

Suitable character graphics can include animals, people, inanimate objects, appliances, natural phenomena, cartoon characters, or the like that can or can not be provided with human features such as arms, legs, facial features or the like. For purposes of enhanced toilet training, it may be desirable for the character graphic to be familiar to the child, such as an identifiable cartoon character. The character graphics should at least be a type that the child can relate to, examples of which could include animals, toys, licensed characters, small appliances, or the like. Character graphics can be made more personable and friendly to the child by including human-like features, human-like expressions, apparel, abilities, or the like. By way of illustration, an animal character graphic can be shown smiling, wearing clothing, playing sports, fishing, driving, playing with toys, or the like. In particular embodiments, the character graphic can desirably be created to project an appearance that could be described as friendly, positive, non-intimidating, silly, independent, inspirational, active, expressive, dauntless and/or persevering.

Throughout this description, the expression “object graphic” denotes a graphic representing an object or thing, which can include an inanimate object or a character.

As used herein, the expression “interactively interrelated” is used to mean that the character graphic is illustrated to be involved in or performing an action or activity, and the object graphic is the object of or is associated with the action or activity. Various forms of interactive interrelationships are disclosed in U.S. Pat. No. 6,297,424. Due to the complicated nature of registering the character graphics and wetness indicator graphics, it is preferred in the invention that the respective graphics are not “interactively interrelated.”

Throughout this disclosure, the term “related” insofar as it refers to the character graphics and wetness indicators refers to the situation where one graphic is the same as or is linked to another graphic. The relationship can be between two or more text messages, between two or more pictorial images, or between a combination of one or more text messages and one or more pictorial images. The general term “graphic” is used herein to mean any design, pattern, or the like that is or becomes visible on an absorbent article, and specifically includes text messages that consist of one or more alphanumeric symbols, pictorial images that consist of one or more pictures, and combinations thereof.

For purposes of illustration only, graphics may be considered “related” where the images are identical; separately illustrate different sizes, shapes, colors of a common object; each illustrate one and the other of two objects that are commonly associated with one another, such as an animal and its paw prints, cartoon character and its foot prints, anthropomorphic character and a related item (e.g., a toaster or vacuum cleaner with human characteristics, and a related outlet plug and the like), a character selected from an animal, cartoon character and anthropomorphic character and its representative foot print, a bug and an outline or silhouette of the bug, the moon and stars, a body of water and water toys, a sandbox and suitable toys, a baseball bat and ball, a barn and animals, or the like. The term “silhouette,” as it is used herein, denotes a completely colored representation of the image, as well as simply an outline of an image. It is preferred in the invention that the related graphics do not illustrate different items used in a particular activity, such as a sporting activity, a gardening activity, a musical activity, and the like. In a similar fashion, two text messages may be considered related in subject matter where the messages: are identical; jointly form a sentence, thought, or action such as “jump” and “up;” each refer to one and the other of two items that are commonly associated with one another; jointly present a question and answer; or the like. Likewise, a text message and a pictorial image may be considered to be related in subject matter where the text names, defines or describes the image; or the like.

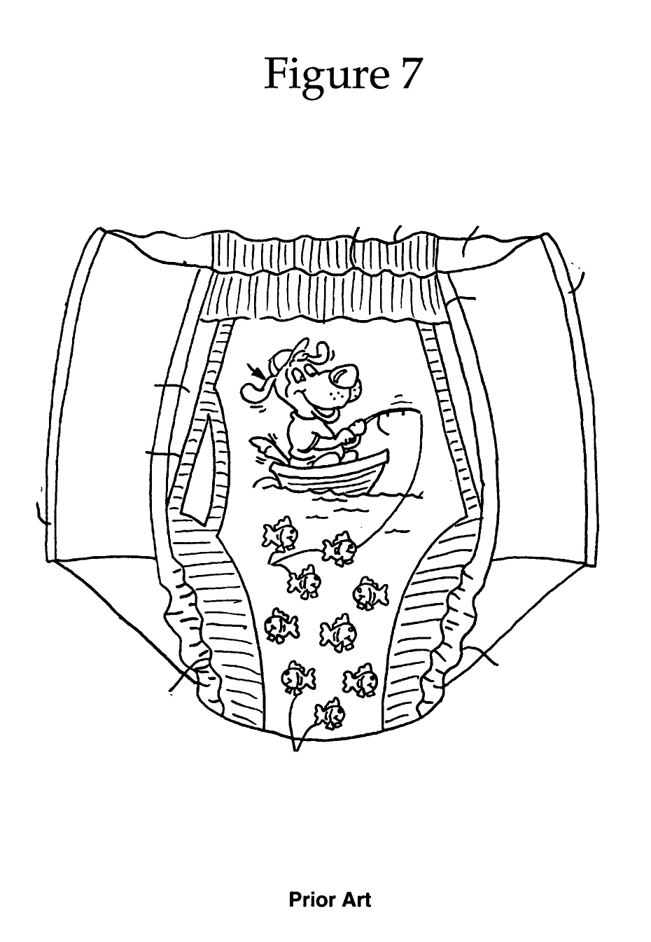

Throughout this description, the term “registered,” insofar as it refers to graphic elements being “registered” on an absorbent article, denotes controlled placement of the graphic on a specific area of the article. For example, a registered character graphic is shown in FIG. 7, whereby the position of dog in the boat is controlled to be in the center of the waist portion, and no other similar character graphics appear in the waist portion. Accordingly, an “unregistered” graphic is a graphic whose precise positioning on the garment has not been controlled. In one embodiment of the invention, the unregistered graphic element is positioned on the article by cutting a web containing a repeating series of similar graphic elements and then placing the web in the waist portion of the article. By cutting and placing the graphic element in this manner, at least one (and sometimes more than one) full character will be visible on the article, whereas other similar characters in the repeating pattern may be cut in half. Another embodiment utilizing an “unregistered” graphic includes directly printing on a component of the absorbent article (e.g., back sheet, chassis layer, etc.), a random series of characters in such a manner that the characters may or may not be centered in the waist portion of the absorbent article. Other mechanisms of placing “unregistered” graphics on an absorbent article are disclosed in U.S. Pat. No. 6,558,499, the disclosure of which is incorporated by reference herein in its entirety.

Embodiments of the invention include one or more of each of the above-mentioned graphics arranged in such a manner that the wetness indicator graphics are visible on the absorbent garment when dry, or when first worn, and then disappear when wet, and in the area of wetness, or disappear over a period of time. Another embodiment includes one in which the wetness indicator graphics are not visible on the absorbent garment when dry, or when first worn, and then appear when wet, and in the area of wetness, or appear over a period of time.

Other embodiments and arrangements of the wetness indicator graphics and character graphics will be readily apparent to those skilled in the art upon reading this description, and consequently, are contemplated in the present invention. For example, the character graphics and the wetness indicator graphics may be positioned anywhere within the absorbent article, and printed on any portion thereof, (e.g., between top sheet and core, on the back sheet, on another material that is positioned on one of the article elements), so long as the graphics are visible through the back sheet of the garment, and so long as the wetness indicator is positioned near the crotch portion (when it is a graphic element that appears or disappears when insulted with urine), and the character is positioned in the waist portion. Alternatively, the wetness indicator graphics may be disposed such that they are visible through the inner lining, as viewed from the inside of the absorbent article.

The present invention relates generally to absorbent articles, and in particular to a pants-type absorbent article that contains a top sheet, a back sheet, and an absorbent core positioned at least partially between the top sheet and the back sheet. We refer to a pants-type absorbent article in this description and the drawings for purposes of illustration only, recognizing that the invention includes all types of absorbent articles, including diapers and adult incontinence products. The absorbent article of the invention preferably has a front waist region, a rear waist region and a crotch region positioned between the front and rear waist regions. The front and rear waist regions are considered “waist portions,” and the crotch region is considered a “crotch portion.” Those skilled in the art recognize that “front” and “rear” in the context of the invention denote for clarity purposes only the front and rear of a user, and that the absorbent garment could be reversed whereby the previously described “front” portion becomes the rear portion, and vice versa.

The article also preferably has a longitudinal centerline extending from the first waist region, through the crotch region and to the second waist region, and a lateral centerline orthogonal to the longitudinal centerline. Thus, the waist portions are positioned adjacent the longitudinal ends of the article, and the crotch portion is positioned near the longitudinal centerline, adjacent the waist portion. The term “adjacent” in the context of the invention does not mean that respective components are in direct contact with, or contiguous with each other. Rather, there may be spaces between the longitudinal ends of the article and the waist portion, and between the waist portion and the crotch portion.

The disposable pants-type absorbent article also may have disposed on an outer surface of the back sheet a disposal fastening mechanism that is capable of maintaining the garment in a rolled up configuration after the garment is rolled up. The disposal fastening mechanism may be a tape, a hook portion of a hook and loop fastener, and may include visible indicia to provide information to the caregiver or wearer of the disposable pants-type absorbent garment, or provide a graphic, or provide indicia indicating the origin of the article.

The invention also encompasses a method of making a disposable pants-type absorbent article having longitudinal end portions and a waist portion adjacent at least one longitudinal end portion. The method includes providing a top sheet material, a back sheet material, and an absorbent core to a garment forming station. The method also includes positioning the absorbent core at least partially between the top sheet material and the back sheet material at the garment forming station. The method further includes providing at least one wetness indicator graphic, and positioning the wetness indicator graphic in the article such that it is visible through the back sheet. In the case of the wetness indicator graphic disappearing or appearing when contacted with urine, the wetness indicator graphic preferably is positioned to be at least partially in contact with the absorbent core. The method further includes providing at least one character graphic and positioning the character graphic in the waist portion. The wetness indicator graphics and character graphics are provided in such a manner that they are related to one another, preferably not interactively interrelated to one another.

Leg elastics preferably are provided along the leg openings for securely holding the leg openings against the thighs of the wearer to improve containment and fit. A pair of stand-up leg gathers or waist containment flaps may be attached to or formed from the body's side surface of the top sheet. Other elastic elements may be disposed in or on the absorbent garment to provide a firmer fit around the tummy and/or waist of the wearer.

The invention now will be described with reference to the attached drawings illustrating preferred embodiments of the invention. For clarity, features that appear in more than one Figure have the same reference number in each Figure. The following description refers to particularly preferred embodiments of the invention, such as the use of specific character graphics and specific wetness indicator graphics that are related to one another. Those skilled in the art will appreciate that the invention is not limited to these specific graphics and that any graphics can be used.

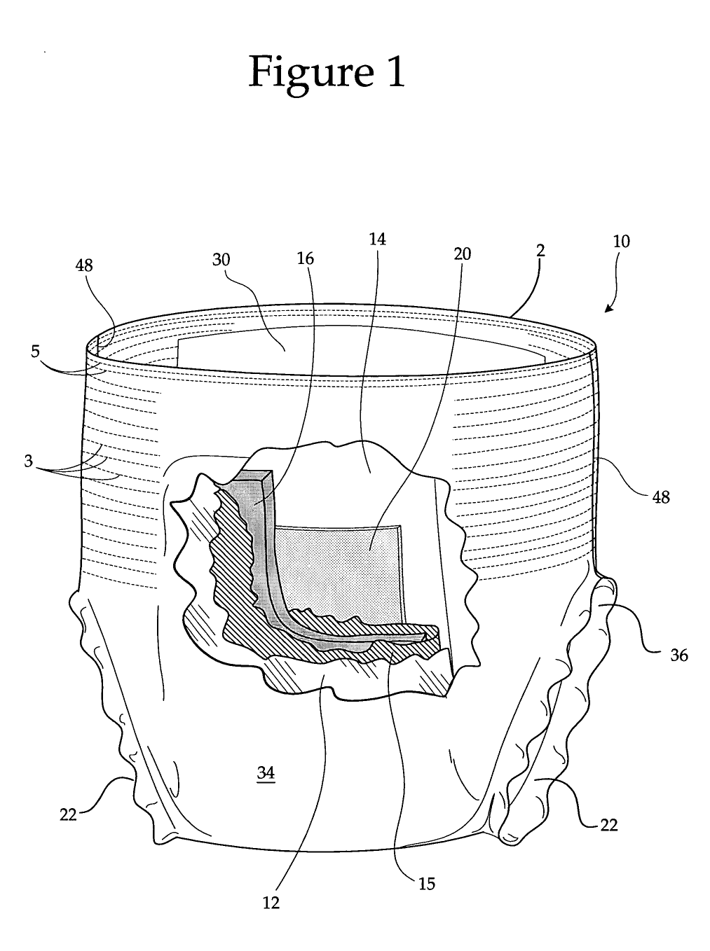

FIG. 1 depicts an embodiment of the present invention as it appears when worn by a user, without any graphics, and with the main body partially cut away to show the absorbent core 16, and other internal components including, for example, an additional layer 20 (e.g., fluid transfer or handling layer, fluid acquisition layer, additional storage layer, wicking layer, and the like), and an optional tissue layer(s) 15 surrounding the core 16. In the depicted embodiment, the garment 10 is comprised of a main body 34 having an exterior facing liquid impervious outer layer 12 or “back sheet,” and a moisture pervious body-contacting inner layer 14 or “top sheet.” An absorbent core 16 is at least partially disposed between the top sheet 14 and the back sheet 12. In the embodiment depicted in FIG. 1 the back sheet 12, top sheet 14, and core 16 comprise the main body 34 of the garment, however in another embodiment of the invention the main body may be made from a separate sheet (for example, a garment chassis) and the back sheet 12, top sheet 14, and core 16 may be assembled separately then attached to the main body.

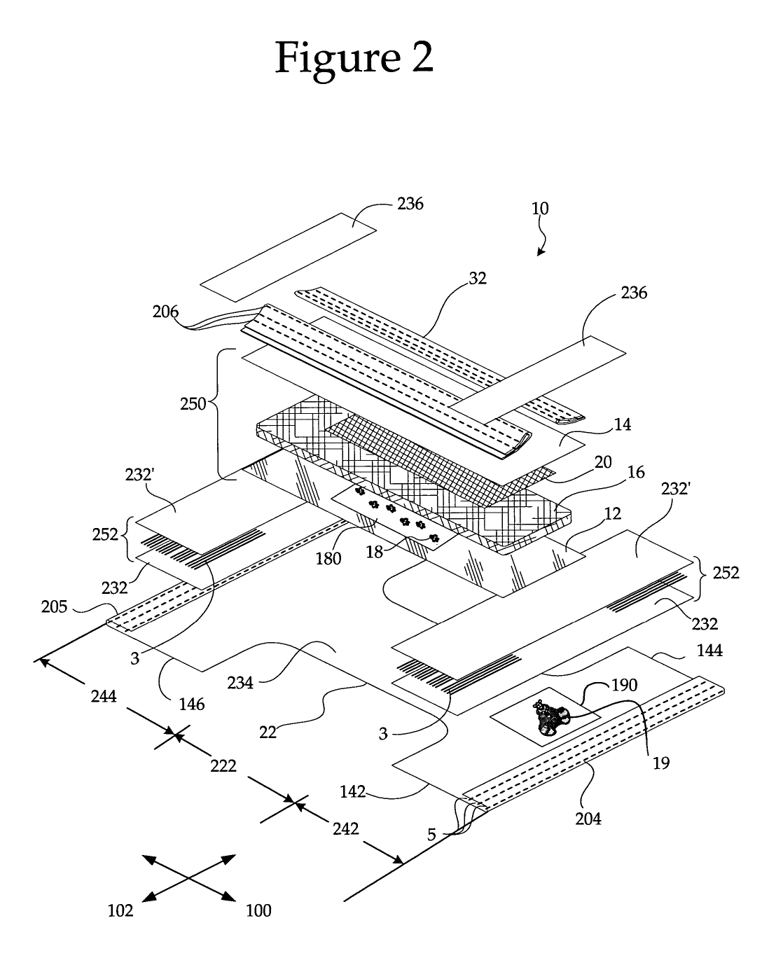

In the embodiment of the present invention depicted in FIG. 1, the garment 10 preferably further comprises various mechanisms for improving the fit of the garment 10 such as leg gathers 36 and standing leg gathers 32 (see, FIG. 2). Such gathers can be used to contract the leg holes 22 around the wearer's legs and body to prevent leakage. A garment 10 of the present invention may also comprise elastic or other fitting devices in the waist portions or other portions of the main body to help contain body exudates.

The various parts of the garment 10 are operatively associated with one another in such a manner that the garment will maintain its desired structure during use. The parts may be operatively associated with one another by a variety of methods known in the art, including, but not limited to: using adhesives such as hot melt adhesives and construction adhesives, chemical or solvent bonding, ultrasonic welding, stitching, heat bonding, or any other method of affixation known or hereafter discovered. All of the parts may be joined to each adjacent part, but some parts may not be joined to others. In one embodiment, the top sheet 14 and back sheet 12 are bonded to one another around their perimeter regions, thereby at least partially encasing and holding the absorbent core 16 in place without having to directly join the absorbent core 16 to any parts of the garment 10. The top sheet 14 or back sheet 12 also may be operatively associated with the absorbent core 16. As understood herein, the term “operatively associated” includes directly joining one part to another, indirectly joining parts together through one or more intermediary parts, whether those intermediary parts are described herein or not, joining parts in such a manner that unjoined parts are captured or held in their proper place, and any other suitable joining mechanism that maintains the structural integrity of the garment 10 for the duration of its use.

FIG. 2 is an exploded view of an embodiment of the present invention with elastic members shown in the elongated position for clarity, and the garment laid flat. The garment 10 has a longitudinal axis 100 corresponding approximately to the rear-to-front axis of the wearer, and a lateral axis 102, orthogonal to the longitudinal axis 100, and corresponding approximately to the side-to-side axis of the wearer. In one embodiment of the invention the lateral axis 102 of the garment 10 is approximately parallel with the machine direction of the garment 10.

In the embodiment of the invention depicted in FIG. 2, the main body 250 of the garment comprises a back sheet 12 and top sheet 14 having substantially identical dimensions. Those skilled in the art will recognize, however, that back sheet 12 and top sheet 14 need not have substantially identical dimensions; rather, either material may be smaller or larger than the other. The main body 250 of the absorbent garment 10 preferably is covered by an outer cover, more preferably, a chassis layer or layers 234. The perimeter of the chassis layer or layers is defined by laterally extending front and rear waist edges 204, 205, longitudinally extending left and right front side edges 142, 144 and left and right rear side edges 146, (right rear side edge not shown), and leg holes 22. The laterally extending front waist edge 204, left and right front side edges 142, 144, and at least part of leg holes 22 form a front waist region 242. The laterally extending rear waist edge 205, left and right rear side edges 146, (right rear side edge not shown), and at least part of leg holes 22 form a rear waist region 244. The remaining portions of leg holes 22 form the crotch region 222. The waist portion is positioned substantially within the front and/or rear waist region 242, 244, and the crotch portion is positioned substantially within the crotch region 222.

Throughout this description, the terms “front,” “rear,” “left,” and “right” merely denote location relationships for purposes of explanation and clarity, and they generally relate to the location depicted in the drawings. Those skilled in the art appreciate that the front and rear of the absorbent garment may be reversed, as well as the left and right sides of the absorbent garment 10 depending upon the vantage point of the viewer.

To form the absorbent garment shown in FIG. 2 into a pants-type absorbent garment of the type shown in FIG. 1, the lateral edge portions 142, 144, 146 may be joined during or after manufacture by any mechanism known in the art or by a combination of such mechanisms. Examples of such mechanisms include: applying adhesives such as hot melt adhesives and construction adhesives, chemical or solvent bonding, stitching, heat bonding, autogenous bonding, ultrasonic bonding, and, preferably, thermal, or heat, bonding. The lateral edge portions 142, 144, 146, also may be held proximal to one another or in an overlapping relationship during use by a fastener, such as a hook-and-loop fastener or adhesive fastener, as are well known in the art. When the lateral edge portions 142, 144, 146 are joined, leg hole cutouts along the lateral edges of the garment 10 form leg holes 22, and the laterally extending waist edges 204, 205 of the garment 110 form a waist encircling edge 2 to form waist hole 30 (FIG. 1). It is particularly preferred that lateral edge portions 142, 144, 146 be joined to one another via an elastically extensible material, or belt, that enables the sides of garment to expand. Alternatively, significant numbers of elastic materials 3 may be disposed near the lateral edges 142, 144, 146 to enable such expansion.

The top sheet 14 and back sheet 12 may be constructed from a wide variety of materials known in the art. The invention is not intended to be limited to any specific materials for these components. The top sheet 14 and back sheet 12 can be shaped and sized according to the requirements of each of the various types of absorbent garment, or to accommodate various user sizes. In an embodiment of the invention in which the garment 10 is a diaper or an adult incontinence brief, the combination of top sheet 14 and back sheet 12, may have an hourglass shape, or may have a rectangular shape, as seen in FIGS. 1 and 2, a trapezoidal “T” shape, or other shape.

Due to the wide variety of backing and liner sheet construction and materials currently available, the invention is not intended to be limited to any specific materials or constructions of these components. The back sheet 12 preferably is made from any suitable pliable liquid-impervious material known in the art. The selection and manufacture of such materials is well known in the art, and is disclosed, for example, in U.S. Pat. No. 6,123,694 issued to Peniak et al., and U.S. Pat. No. 6,176,952 issued to Maugans et al., the disclosure of each of which is incorporated herein by reference in its entirety. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 12 may be pigmented with, for example, titanium dioxide, to provide the garment 10 with a pleasing color or to render the back sheet 12 opaque enough that exudates being contained by the garment 10 are not visible from outside the garment, but yet transparent enough to allow graphic 18 to be visible. In addition, the back sheet 12 may be formed in such a manner that it is opaque, for example, by using various inert components in the polymeric film and then biaxially stretching the film. Other back sheet materials will be readily apparent to those skilled in the art. The back sheet 12 preferably has sufficient liquid imperviousness to prevent any leakage of fluids. The required level of liquid imperviousness may vary between different locations on the garment 10.

The back sheet 12 may further comprise separate regions having different properties. In one embodiment, portions of the back sheet 12 are air-permeable to improve the breathability, and therefore comfort, of the garment 10. The different regions may be formed by making the back sheet 12 a composite of different sheet materials, chemical treatment, heat treatment, or other processes or methods known in the art. Some regions of the back sheet 12 may be fluid pervious. In one embodiment of the invention, the back sheet 12 is fluid impervious in the crotch region 222, but is fluid pervious in portions of the first and second waist regions 242, 244. The back sheet 12 may also be made from a laminate of overlaid sheets of material. It is preferred that the back sheet 12 be fluid impermeable with no differing regions of air permeability.

The moisture-pervious top sheet 14 can be comprised of any suitable relatively liquid-pervious material known in the art that permits passage of liquid there through. Non-woven liner sheet materials are exemplary because such materials readily allow the passage of liquids to the underlying absorbent core 16. Examples of suitable liner sheet materials include non-woven webs of polypropylene, polyethylene, nylon, polyester and blends of these materials. Other suitable materials include apertured webs, as well as laminates of nonwoven materials, such as a spunbond-meltblown-spunbond (SMS) laminate.

The back sheet 12 may be covered with a fibrous, non-woven fabric chassis layer(s) 234 (or “cover sheet” 234) such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded non-woven web of synthetic fibers such as polypropylene, polyethylene or polyester fibers; a non-woven web of cellulosic fibers, textile fibers such as rayon fibers, cotton and the like, or a blend of cellulosic and textile fibers; a spun-bonded non-woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers.

The cover sheet 234 also may comprise a non-woven polyethylene or polypropylene sheet, a polyethylene film, or any other suitable garment material known in the art or hereafter discovered. All or part of the chassis layer 234 may comprise a liquid pervious or liquid impervious material or a may be zone-treated to be partially liquid pervious or impervious. The chassis layer 234 may be stretched in one or more directions during the manufacturing process, thereby reducing its elasticity in the direction of stretch.

Alternatively, the back sheet 12 may comprise three panels wherein a central poly back sheet panel is positioned closest to absorbent core 16 while outboard non-woven breathable side back sheet panels are attached to the side edges of the central poly back sheet panel. Alternatively, the back sheet 12 may be formed from microporous poly coverstock for added breathability.

Although not illustrated in the drawings, the top sheet 14 may be formed of three separate portions or panels. Such an embodiment is disclosed, for example, in U.S. Pat. No. 5,275,590 issued to Huffman et al., which is incorporated herein by reference in its entirety, and in a manner consistent with the present invention. Those skilled in the art will recognize, however, that top sheet 14 need not be made of three separate panels, and that it may be comprised of one unitary item, or of a top sheet material 14, with separate standing leg gathers material 32 attached thereto, as shown in FIG. 2. A first top sheet panel may comprise a central top sheet panel formed from preferably a liquid-pervious material that is either hydrophobic or hydrophilic. The central top sheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel is a cover stock of single ply non-woven material which may be made of carded fibers, either adhesively or thermally bonded, perforated plastic film, spunbonded fibers, or water entangled fibers, which generally weigh from about 10 to about 25 g/m². and have appropriate and effective machine direction and cross-machine direction strength suitable for use as a baby diaper cover stock material. The central top sheet panel preferably extends from substantially the second waist region 244 to the first waist region 242, or a portion thereof.

The second and third top sheet panels (e.g., outer top sheet panels), in this alternative embodiment may be positioned laterally outside of the central top sheet panel. The outer top sheet panels preferably are substantially liquid-impervious and hydrophobic, preferably at least in the crotch area. The outer edges of the outer top sheet panels may substantially follow the corresponding outer perimeter of the back sheet 12. The material for the outer top sheet portions or panels preferably is polypropylene and can be woven, non-woven, spun bonded, carded or the like, depending on the application.

The respective top sheet panels may be attached to one another by, e.g., an adhesive. At the point of connection with the outer edges of the central top sheet panel and the inner edges of the outer top sheet panels extend upwardly to form waste containment flaps 32 (waist containment flaps 32, or standing leg gathers 32, are shown as separate elements in FIG. 2, but they need not be). The waste containment flaps 32 preferably are formed of the same material as the outer top sheet panels, as in the embodiment shown. They most preferably are an extension of the outer top sheet panels.

The waste containment flaps 32 may be treated with a suitable surfactant to modify their hydrophobicity/hydrophilicity as desired, and they may be treated with skin wellness ingredients to reduce skin irritation. Alternatively, the waste containment flaps 32 may be formed as separate elements and then attached to the body side liner or top sheet 14. In this alternative embodiment, the central top sheet panel may extend past the connection point with the waste containment flaps 34, and even extend to the periphery of the back sheet 12.

The waste containment flaps 32 preferably include a portion that folds over onto itself to form a small enclosure. At least one, and depending on the size of the enclosure sometimes more than one, elastic element 206 may be secured in the enclosure in a stretched condition. As is well known in the art, when the flap elastic elements 206 attempt to assume the relaxed, unstretched condition, the waste containment flaps 32 rise above the surface of the center of the top sheet 14, as shown in FIG. 2.

The top sheet 14 may be made of any suitable relatively liquid-pervious material currently known in the art or later discovered that permits passage of a liquid there through. Examples of suitable top sheet materials include non-woven spun-bonded or carded webs of polypropylene, polyethylene, nylon, polyester and blends of these materials, perforated, apertured, or reticulated films, and the like. Non-woven materials are exemplary because such materials readily allow the passage of liquids to the underlying absorbent core 16. The top sheet 14 preferably comprises a single-ply non-woven material that may be made of carded fibers, either adhesively or thermally bonded, spun bonded fibers, or water entangled fibers, which generally have a basis weight of from about 10 to about 25 g/m², and have appropriate and effective machine direction (longitudinal) and cross-machine (lateral) direction strength suitable for use as a top sheet material for the given application. The present invention is not intended to be limited to any particular material for the top sheet 14, and other top sheet materials will be readily apparent to those skilled in the art.

The top sheet 14 may further comprise several regions having different properties. In one embodiment of the present invention, the laterally distal portions of the top sheet 14, especially those used to make second and third top sheet panels, preferably are substantially fluid impervious and hydrophobic, while the remainder of the top sheet 14 (e.g., central top sheet panel) is hydrophilic and fluid pervious. Different top sheet properties, such as fluid perviousness and hydrophobicity, may be imparted upon the top sheet 14 by treating the top sheet 14 with adhesives, surfactants, or other chemicals, using a composite of different materials, or by other means. This type of top sheet 14 material typically is referred to as a zone treated top sheet, or three zone top sheet. The top sheet 14 also may be made from a laminate of overlaid sheets of material. The top sheet 14 also may be treated in specific areas like the crotch region, with skin wellness ingredients such as aloe, vitamin E, and the like.

As noted elsewhere herein, the top sheet 14 and back sheet 12 may be substantially coterminous, or they may have different shapes and sizes. The particular design of the top sheet 14 and back sheet 12 may be dictated by manufacturing considerations, cost considerations, and performance considerations. Preferably, the top sheet 14 is large enough to completely cover the absorbent core 16, and the back sheet 12 is large enough to prevent leakage from the garment 10. The design of top sheet 14 and back sheet 12 is known in the art, and a skilled artisan will be able to produce an appropriate top sheet 14 and an appropriate back sheet 12 without undue experimentation.

The top sheet 14 and the back sheet 12 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesiveis used to join the top sheet 14 to the back sheet 12. The particular joining method may be dictated by the types of materials selected for the top sheet 14 and back sheet 12.

In one embodiment of the present invention, the top sheet 14 is operatively associated with the back sheet 12 around the perimeter of the top sheet 14. In this embodiment, the top sheet 14 and back sheet 12 may be operatively associated with one another by using hot melt adhesives, ultrasonic bonding, or any other suitable method known in the art. Also in this embodiment, the top sheet 14 and back sheet 12 may be bonded to one another in substantially all areas not having intermediately placed parts, such that some or all of the intermediately placed, or “sandwiched,” parts are physically captured between the top sheet 14 and back sheet 12, but not bonded to the back sheet 12 or top sheet 14.

An absorbent core 16 preferably is disposed between the inner surfaces of the back sheet 12 and the top sheet 14. The absorbent core 16 may be comprised of one or more layers of material, such as an absorbent layer for storing fluids and an acquisition layer for distributing fluids. Such multiple layer absorbent cores are known in the art and disclosed in U.S. Pat. No. 5,439,458 issued to Noel et al., which is incorporated herein by reference in its entirety, and in a manner consistent with the present invention.

The absorbent core 16 may be made from any absorbent material or materials known in the art. In one embodiment of the invention, the absorbent core 16 comprises wood fibers or other fibers such as tow fibers, chemical wood pulp, or any other suitable liquid absorbing material, such as commercially available fluff pulp or fluffed bleached kraft softwood pulp. In another embodiment of the invention, the absorbent core 16 comprises a combination of a porous fibrous web and super absorbent particles. Such absorbent cores are known in the art and are disclosed, for example, in U.S. Pat. No. 5,281,207 issued to Chmielewski et al., which is incorporated herein by reference in its entirety. In such an embodiment, the absorbent core 16 may be surrounded by a liquid pervious tissue over-wrap 15 (FIG. 1), or other material.

The absorbent core 16 also may be a laminate material, as described in U.S. Pat. No. 6,068,620, the disclosure of which is incorporated by reference herein in its entirety. Such laminate materials typically include outer tissue layers 15 (FIG. 1) surrounding a central fibrous layer 16 that contains a high percentage by weight of superabsorbent polymer (SAP). Any type of SAP can be used in this embodiment, or in any absorbent core 16 that is useful in this invention. The SAP generally is a water-insoluble but water-swellable polymeric substance capable of absorbing water in an amount which is at least ten times the weight of the substance in its dry form. In one type of superabsorbent material, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Any SAP now known or later discovered can be used in the absorbent core 16 of the invention. Commercially available SAPs include a starch modified superabsorbent polymer available under the trade name SANWET® or HYSORB® from BASF Corporation, Portsmouth, Va. SANWET® is a starch grafted polyacrylate sodium salt. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP manufactured by Seitetsu Kagaku Co., Ltd.; ARASORB manufactured by Arakawa Chemical (U.S.A.) Inc.; ARIDALL 1125 manufactured by Chemdall Corporation; FAVOR manufactured by Stockhausen Inc.; AQUA KEEP SA60S, manufactured by Seitetsu Kagaku Co., Ltd.; DIAWET, commercially available from Mitsubishi Chemicals, Japan; FLOSORB, available from SNF Floerger, France, AQUALIC, available from Nippon Shokubai, Osaka, Japan.

The SAP may be provided in any particle size, and suitable particle sizes vary greatly depending on the ultimate properties desired. For example, a fine particulate rather than a coarse particulate may be used in the invention, and preferably a fine particulate that passes through an about 200 mesh screen may be used.

The absorbent core 16 may be surrounded by a liquid pervious tissue over-wrap 15 (FIG. 1), or other material(s), which may be treated to be hydrophobic or hydrophilic, or to have other properties. The absorbent core 16, and any tissue wrap enclosing it, may be folded, crimped, thermally bonded, or otherwise manipulated to provide additional benefits. It is envisioned that a variety of folding patterns may be employed to provide additional fluid handling capabilities. For example, the absorbent core 16 may be folded into a U shape, a C shape, a G shape, a Z shape, or other shapes, as viewed along the longitudinal axis 100, to provide fluid handling channels, multiple layers of absorbent material, or other benefits. Folded absorbent cores are discussed, for example, in U.S. Pat. No. 6,068,620.

The absorbent core 16 generally is elongated along the longitudinal axis 100 of the garment, and may extend along either or both of the lateral axis 102 and the longitudinal axis 100 to the outer perimeter of the garment. In the embodiment depicted in FIGS. 1 and 2, the absorbent core 16 is substantially rectangular in shape, however, it also may have rounded ends or other shapes, such as an “I” shape or a “T” shape. The absorbent core 16 also may have channels, grooves or pockets, and may have a varying thickness. In an embodiment having a channeled or pocketed absorbent core 16, such channels or pockets may be substantially vacant, or may be filled with additional SAP or additional supplemental absorbent cores having similar or different properties than the absorbent core 16.

As mentioned previously, the various parts of the garment 10 preferably are operatively associated with one another in such a manner that the garment will maintain its desired structure during use. The parts may be operatively associated with one another by a variety of methods known in the art, including, but not limited to: using adhesives such as hot melt adhesives and construction adhesives, chemical or solvent bonding, ultrasonic welding, stitching, heat bonding, autogenous bonding, or any other method of affixation known or hereafter discovered. U.S. Pat. No. 4,919,738 issued to Ball et. al. discloses a method of autogenous bonding, and its disclosure is herein incorporated by reference in its entirety in a manner consistent with the invention. All of the parts may be joined to each adjacent part, but some parts may not be joined to others.

The core assembly 250 may comprise additional layers 20 of material that may reduce rewet of the top sheet 14, reduce strikethrough times or otherwise improve the absorbency, dryness and other properties of the garment 10. Examples of the one or more additional layers 20 include any layer selected from a fluid acquisition layer, a distribution layer, an additional fibrous layer optionally containing SAP, a wicking layer, a storage layer, a dryness layer, a softness layer, or combinations and fragments of these layers. Such layers may be provided to assist with transferring fluids to the absorbent core 16, handling fluid surges, preventing rewet, containing absorbent material, improving core stability, or for other purposes. Skilled artisans are familiar with the various additional layers 20 that may be included in an absorbent article, and the present invention is not intended on being limited to any particular type of materials used for those layers. Rather, the invention encompasses all types of wicking layers, all types of distribution layers, etc., to the extent that type of layer is utilized.

The core assembly 250 may be attached to the cover sheet 234 by any mechanism known in the art, such as by ultrasonic bonding or by the use of lines of hot melt adhesive. The bond between the core assembly 250 and the cover sheet 234 may be reinforced by laterally-extending end strips 236 that preferably are applied over the longitudinal ends of the core assembly 250 and bonded to the underlying structure of the garment 10. The end strips 236 also may hold the ends of the standing leg gathers 32 so that the standing leg gathers 32 face inward or outward. Such end strips 236 preferably comprise a fluid pervious non-woven material, but may be fluid impervious or a material other than a non-woven material. Such materials are known in the art. The end strips 236 also may help prevent the longitudinal flow of exudates past the ends of the core assembly 250, particularly if the edges of the non-woven strips overlying the core assembly 250 are left un-bonded so that they form pockets to hold exudates.

Waist elastics 5 and tummy elastics 3 may be incorporated into the garment 10 to contract the garment 10 about the wearer's abdomen. Such elastics are typically stretched as they are joined to the garment 10 so that the contraction of the elastics causes the garment 10 to contract about the wearer. The elastics also may be applied in an unstretched state and then mechanically stretched to create an elasticized region in the garment (often called a zero-strain laminate). The elastics also may be applied in an inelastic state and then heat activated to cause them to become elasticized. The elastics 3, 5, and 206 may be made from natural or synthetic rubber, elastomers, LYCRA® elastomer (available from E.I. DuPont de Nemours and Company, a business having offices in Wilmington, Del.), polyurethane, heat shrinkable polymer ribbons, or any other suitable elastic material or composite. Such materials are known in the art.

In a preferred embodiment, the waist elastics 5 are located proximal to one or both laterally extending edges 204, 205 of the cover sheet 234, and are thereby located along the waist encircling edge of the fully assembled garment 10. In such an embodiment, the waist elastics 5 may be located on one side of the cover sheet 234, within a fold in the cover sheet 234 (as shown in FIG. 2), or otherwise fixed in the proximity of the laterally extending waist edges 204, 205. U.S. Pat. No. 4,515,595 issued to Kievit et. al. and U.S. Pat. No. 4,816,025 issued to Foreman illustrate other embodiments of elasticized waist features of absorbent garments, and are hereby incorporated by reference in their entirety.

Tummy elastics 3 also may be disposed in the garment 10 between the laterally extending waist edges 204, 205 and the leg opening cutouts 22 to thereby be positioned across the wearer's stomach. The tummy elastics 3 may be attached directly to the cover sheet 234 or may be disposed between a pair of carrier layers 232, 232′ to form tummy elastic assemblies 252 that are attached to the cover sheet 234. The tummy elastics 3 may be located on the interior or exterior side of the cover sheet 234, and may be covered by additional layers of material. In a preferred embodiment, the tummy elastics 3 are affixed between a pair of carrier layers 232, 232′. The carrier layers 232, 232′ preferably comprise non-woven materials, but may be made of any suitable material, and may be liquid pervious or liquid impervious. The carrier layers 232, 232′ are preferably gas pervious to allow the garment 10 to “breathe.”

In one embodiment, the tummy elastics 3 may extend across the entire width of the garment 10. In a preferred embodiment, shown in FIGS. 1 and 2, the tummy elastics 3 extend across the lateral sides of the garment 10, but not across the portion of the garment 10 overlying the absorbent core 16. Such a preferred embodiment may provide improved fit and comfort and improve the garment's appearance. U.S. Pat. No. 5,449,353 issued to Watanabe et. al. and U.S. Pat. No. 5,749,865 issued to Yamamoto et al. illustrate other embodiments of elasticized waist features of absorbent garments, and are incorporated herein by reference in their entirety.

The elastics 3, 5, 206 or any other elastics may be joined to the garment 10 by the use of a flexible adhesive or other suitable joining method. Suitable adhesives include HL-1258 by H.B. Fuller Company of St. Paul, Minn.; Findley 2031 and H2587-01 by Ato Findley Inc. of Wauwatosa, Wis.; and DISPOMELT® 34-5665 by National Starch Co. of Bridgewater, N.J. Adhesives that may be used to secure elastic elements to the absorbent garment include DISPOMELT® 34-578A by National Starch Co. of Bridgewater, N.J. In a preferred embodiment of the invention, the adhesive utilized includes HL 1486UZP, which is available from H.B. Fuller Company of St. Paul, Minn. This and other methods for attaching elastics to absorbent garments are known in the art.

As noted previously, it often is desirable for an absorbent garment to contract around various parts of the wearer's body to provide improved comfort and exudate containment. In a preferred embodiment of the present invention depicted in FIG. 2, the garment 10 further comprises at least one standing leg gather 32, and preferably at least 2 standing leg gathers 32, for improving the ability of the garment 10 to contain body exudates. The standing leg gathers 32 may be formed by incorporating a plurality of gather elastics 206 into folds in the top sheet 14 (not shown), or preferably may be provided as separate standing leg gather assemblies that are attached to the garment 10 near the leg hole cutouts 22 as shown in FIG. 2. The gather elastics 206 cause the standing leg gathers 32 to rise above the interior surface of the garment 10, thereby forming vertical curtains of material that help contain exudates. The standing leg gathers 32 may be liquid pervious or liquid impervious, and more than one pair of opposing standing leg gathers 32 may be provided.

Additional elastics (not shown) also may be incorporated into the cover sheet 234, top sheet 14 or back sheet 12 adjacent the leg hole cutouts 22 to form non-standing leg gathers 36 (FIG. 1), as is known in the art. Non-standing leg gathers 36 contract the garment 10 around the wearer's legs and body to prevent leakage. U.S. Pat. Nos. 3,860,003 and 4,081,301 issued to Buell, U.S. Pat. No. 4,695,278 issued to Lawson, U.S. Pat. No. 4,808,177 issued to Des Marais, U.S. Pat. No. 4,795,454 issued to Dragoo, and U.S. Pat. No. 4,938,755 issued to Foreman illustrate other embodiments of leg cuffs and gathers in absorbent garments, and the disclosures of these patents are hereby incorporated by reference in their entirety.

The absorbent garment 10 of the invention also preferably includes a fastening mechanism by which the front waist region 242 is associated with rear waist region 244 to form a waist hole 30. The fastening mechanism may comprise a permanent seal whereby the respective side edges 142, 144, 146 are attached to one another to form side seals 48 (FIG. 1). The absorbent garment 10 then can be pulled on and off like an undergarment or pair of pants. Any mechanism can be used to form the permanent seal 48. The use of the expression “permanent seal” in this context is not meant to encompass seals that cannot be broken, but rather permanent is meant to encompass sealing mechanisms that are not intended to be broken during normal use and application. The respective side edges 142, 144, 146 can be associated with one another to form seal 48 using techniques known in the art, including, for example, using adhesives such as hot melt adhesives and construction adhesives, chemical or solvent bonding, ultrasonic welding, stitching, heat bonding, or any other method of affixation known or hereafter discovered.

The absorbent garment 10 of the invention may also include a releasable fastening mechanism by which the front waist region 242 is associated with rear waist region 244 to form a waist hole 30. The releasable fastening mechanism may comprise a mechanism whereby the respective side edges 142, 144, 146 are releasably attached to one another to form side seals 48 (FIG. 1). Releasable fastening mechanisms are well known in the art, (especially when used on diapers), and may include tabs laterally extending from the laterally opposing rear side edges 142, 144, 146, adhesive strips, belts, and the like. The particular mechanism by which the respective parts of garment 10 may be adhered to one another include hook and loop type fasteners, pressure sensitive adhesives, snaps, clips, pins, and the like. Those skilled in the art are capable of manufacturing garment 10 to have either permanent or releasable fastening mechanisms, using the guidelines provided herein.

The wetness indicator graphics 18 of the present invention preferably are in fluid communication with the absorbent core 16, and yet are visible through back sheet material 12. These wetness indicator graphics 18 preferably are disappearing graphics. One manner of arranging the respective indicator graphics 18 is to imprint them on a separate web or multiple webs of material 180, which can be any type of material (e.g. polyolefin film, nonwoven, tissue and the like.) An embodiment of the invention includes imprinting the wetness indicator graphics 18 directly on the absorbent core facing surface of the back sheet 12. Other graphic elements, such as indicia surrounding the leg holes, and the like, also can be positioned together with the wetness indicator graphics 18.

In yet another alternative embodiment of the invention, the wetness indicator graphics 18 are not graphic elements that disappear when wetted, but rather disappear over time. In this embodiment, the wetness indicating graphics 18 could be positioned anywhere in the article. They do not need to be in fluid communication with the absorbent core.

The character graphics 19 of embodiments of the invention preferably are positioned in a waist region 242, 244 of the article, and more preferably a waist portion that is positioned adjacent the longitudinal end 204 of the article. The character graphics 19 may be positioned anywhere on the article so long as it can be viewed when the article is worn. One manner of arranging the character graphics 19 is to imprint them on a separate web or multiple webs of material 190, which can be any type of material (e.g. polyolefin film, nonwoven, tissue and the like.) An embodiment of the invention also includes imprinting the character graphics 19 directly on the cover sheet 234, or back sheet 12, or carrier layer 232, 232′.

When respective graphic elements are imprinted on a separate web(s) 180, 190, the web(s) 180, 190 can be positioned on the absorbent article 10 in any suitable manner. For example, the web(s) 180, 190 can be positioned on the absorbent article 10 in its respective position using adhesives, ultrasonic or thermal bonding, or it may be friction fitted into place. It is preferred that web(s) 180, 190 be placed on absorbent article 10 using cut-and-place techniques that are well known in the art, and then placing the web(s) 180, 190 on a specific portion of the article 10. It is known to effect synchronous, in-line placement of absorbent core pads on a continuous web of material, as described in U.S. Pat. No. 5,415,716, the disclosure of which is incorporated by reference herein in its entirety. It further is known that graphics can be applied in registration on a moving sheet of material that ultimately is used in an absorbent garment by controlling the timing of placing the discrete graphic material. A number of documents describe placing graphic materials in registration on a moving web, including U.S. Pat. Nos. 6,165,306, 6,149,755, 6,095,218, 6,074,333, and 6,059,710, the disclosures of each of which are incorporated by reference herein in their entireties. Any of the methods and apparatus described in these documents can be used in the invention to place the web(s) 180, 190 on the absorbent garment 10.

A preferred embodiment of the invention involves printing a series of character graphics 19 on a web 190. The web 190 then can be cut to a desired size and placed in the waist portion of the article 10 as it is being formed. This embodiment negates the need to register any particular graphic 19 on the article, since the web 190 will include a plurality of character graphics in differing positions. For example, web 190 may include a plurality of characters from a publication, book, magazine, movie, or television show, such as the Bear in the Big Blue House® or a plurality of characters such as bugs. The plurality of character graphics 19 can be sized, and web 190 cut in a size sufficient to ensure that at least one complete character graphic 19 is placed on the article without having to use a complicated registration procedure.

In an alternative embodiment, if a character graphic 19 is registered on the absorbent article 10, it is especially preferred that the wetness indicator graphics 18 are related to the character graphic 19, but not related to any particular activity that the character graphic may be involved in. For example, if a registered animal playing an instrument were used as the character graphic 19, the wetness indicator graphic 18 would be related to the animal in some fashion (e.g., paw prints, animal ears, a silhouette of the animal, etc.), but not related to the instrument or music (e.g., not a musical note, silhouette of the instrument, etc.). Other embodiments that are less preferred, however, include registered character graphics 19 partaking in an activity and wetness indicator graphics 18 that are related to that activity.

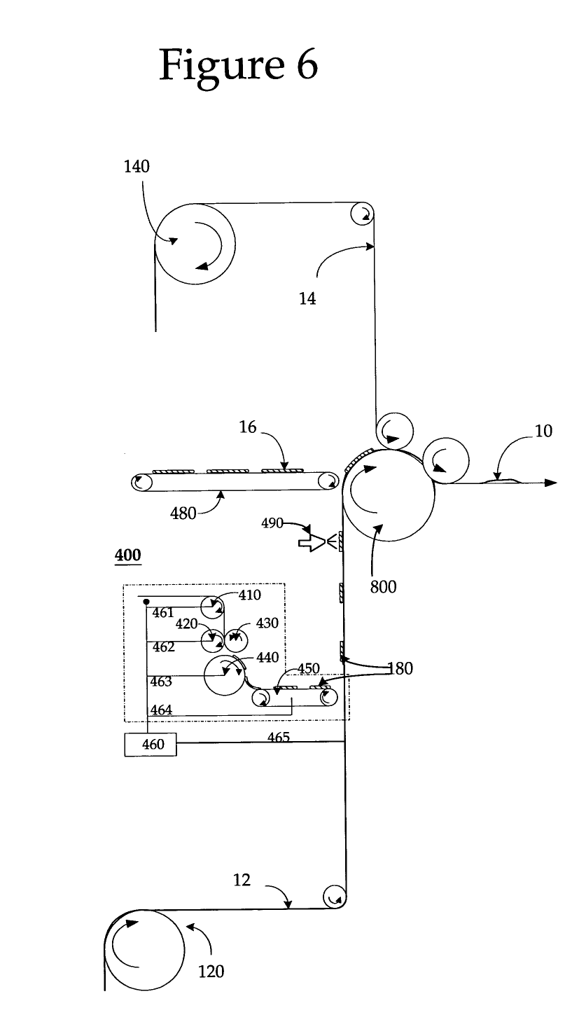

The methods and apparatus suitable for placing web 180 (or web 190—referred to collectively in this discussion only as web 180) can include any cut-and-place apparatus capable of cutting a material from a moving web at a first speed, and placing it on another moving web at a second speed that may be same as or different from the first speed. Suitable cut-and-place apparatus are disclosed in, inter alia, U.S. Pat. Nos. 6,165,306, 6,149,755, 6,095,218, 6,074,333, 6,059,710, and 5,415,716, the disclosures of each of which are incorporated by reference herein in their entireties. A simplified cut-and-place apparatus is illustrated in FIG. 6 and includes a feed roller system 410 that feeds a web of material 180 containing the graphic at a first speed to the cut-and-place apparatus 400. Those skilled in the art will recognize that feed roller system 410 may be comprised of any number of component parts, such as a supply reel, a plurality of dancer rolls and edge guide rolls, and pull rolls to establish the first speed of the web material.

Feed roller system 410 feeds the web of material to a cutting station, illustrated in FIG. 6 as a pair of rollers in cutting engagement with one another. Any type of cutting device can be used in the present invention that is capable of controlled cutting of a moving web of material. FIG. 6 shows a rotatable knife roll 420 in cutting engagement with a rotatable anvil roll 430. The knife roll can be controlled to control cutting the web at the appropriate locations to form an appropriately sized web 180.

Upon cutting, the web 180 is placed on a rotatable vacuum transfer drum 440 that alters the velocity of the moving web to the second speed so as to control its placement on, for example, an inner surface of, for example, back sheet 12, which is moving at a speed different from the speed at which the web 180 containing the graphics is fed to cut-and-place apparatus 400. FIG. 6 illustrates an embodiment where the web 180 first is transferred to back sheet 12, and then placed in registration on absorbent article 10 at placement forming station 800. Moving web 12 also may be chassis layer(s) 234, or any other portion of the article 10, as will be appreciated by those skilled in the art.

The rotatable vacuum transfer drum 440 can be any type of rotating drum capable of drawing a vacuum so that it can grab onto and hold the now severed web 180, and ultimately transfer it to absorbent article 10 via conveyor 450, and back sheet 12, for example. In this embodiment, conveyor 450 transports the web 180 at the second speed to the moving back sheet 12, and preferably, the speed of the web that contains absorbent article 10. Skilled artisans will appreciate, however, that rotatable vacuum transfer drum 440 could deposit the web 180 on a suitable surface of back sheet 12 or absorbent core 16, without the use of the conveyor 450.

The cut-and-place apparatus 400 can be controlled by controller 460. Controller 460 monitors and controls the first speed at which the moving web is conveyed by monitoring and controlling feed roller system 410 via control 461, and by monitoring and controlling knife roll 420 and anvil roll 430 via control 462. Controller 460 also monitors and controls the second speed at which the severed web 180 is conveyed by monitoring and controlling the rotatable vacuum transfer drum 440 via control 463, the conveyor 450 via control 464, and by monitoring the speed of moving back sheet 12 via control 465. Those skilled in the art will recognize that various modifications may be made to controller 460 to adequately control the first and second speeds such that the web 180 is accurately placed on an appropriate surface of back sheet 12, absorbent core 16, or other elements of absorbent article 10. Using the guidelines provided herein, those skilled in the art are capable of designing a suitable controller 460 without undue experimentation.

FIG. 6 illustrates the web 180 containing the wetness indicator graphics 18 being positioned on an interior surface of the back sheet 12, but the invention is by no means limited to this particularly preferred embodiment and configuration. The web 180 could be positioned on a back sheet facing surface of absorbent core 16. In addition, two or more webs 180 could be positioned on either of the aforementioned surfaces of the back sheet 12 and absorbent core 16, using additional cut-and-place apparatus.

The embodiment shown in FIG. 6 illustrates an adhesive applicator 490 applying adhesive to web 180 to ensure its attachment to absorbent article 10. Application of adhesive via applicator 490 is optional in the present invention. Adhesive could be applied to either or both, or neither surface of web (i.e., absorbent core facing surface and back sheet facing surface).

Any mechanism 490 capable of supplying an adhesive, albeit a spray adhesive, or one that is “rubbed” on, can be used in the invention, such as an extrusion applicator, a stencil applicator, or a printing applicator. Suitable adhesives include any adhesive commonly employed in absorbent garments that is useful in adhering one or more components to together. It is particularly preferred to use construction adhesives, including HL-1258 by H. B. Fuller Company of St. Paul, Minn.; Findley 2031 and H2587-01 by Ato Findley Inc. of Wauwatosa, Wis.; and DISPOMELT® 34-5665 by National Starch Co. of Bridgewater, N.J. Other adhesives that may be used in the invention include DISPOMELT® 34-578A, available from National Starch Co. of Bridgewater, N.J. Any of these adhesives may be used in all adhesive applications in the absorbent garment, or only in select applications as a construction adhesive for bonding parts of the garment as the top sheet, back sheet, absorbent core, and additional layer(s). Other adhesives also may be used.

It is preferred in the invention that article 10 be formed at garment forming station 800 where web 180 is positioned at least partially between the absorbent core 16 and the back sheet 12. It will be appreciated, however, that web 180 could be positioned on the back sheet facing surface of absorbent core 16, or on an additional layer(s) (not shown) and then secured to the remaining elements of article 10 at garment forming station 800, using the techniques described herein.

The absorbent core 16 of the invention preferably is used immediately after it is formed as a component part of the absorbent article 10. In this context, the absorbent cores 16 would be transported to garment forming station 800 via core conveyor 480 (or any other conveying device) where they will be positioned at least partially between a top sheet 14 and a back sheet 12. Top sheet material 14 may be supplied to forming station 800 by top sheet supply mechanism 140, which can be any supply mechanism capable of supplying top sheet 14 to garment forming station 800. Preferably, top sheet material 14 is supplied via a supply roller 140 and select feed or guide rollers. Back sheet material 12 likewise can be supplied to forming station 800 by back sheet supply mechanism 120, which can be any supply mechanism capable of supplying back sheet 12 to garment forming station 800. Preferably, back sheet material 12 is supplied via a supply roller 120 and select feed or guide rollers. Forming station 800 brings together the respective components of absorbent article 10 by positioning absorbent core 16 at least partially between top sheet material 14, and back sheet material 12. After placing a disposal fastening mechanism on article 10, the final absorbent article 10 then may be cut and folded to the appropriate size and shape downstream from forming station 800.

The embodiment illustrated in FIG. 6 could include a number of additional devices used in preparing absorbent garments. For example, an additional cut-and-place apparatus could be used to position web 190 on any one of the components of absorbent article 10. In addition, it is conventional to employ leg elastics 36, and standing leg gathers 32 (FIGS. 1 and 2). Leg elastics 36 could be supplied to garment forming station 800 by any suitable manner known in the art. It is conventional to apply adhesives to either the leg elastics 36 themselves, or to apply adhesives on back sheet 12 or top sheet 14, and then dispose the leg elastics 36 there between. The leg elastics 36 may be supplied as an elastic element, or as a laminate of elastic elements disposed between two outer layers, preferably outer non-woven materials.

Standing leg gathers 32 can be applied as a separate material to top sheet 14, and disposed on the exterior facing side (e.g., body facing side) of the top sheet 14. The standing leg gathers 32 can be applied either prior to, or downstream from, garment forming station 800 using techniques known in the art. The standing leg gathers 32 may be secured in place on garment 10 by placing outer sheets, preferably, outer non-woven sheets 236 over the longitudinal end portions of the standing leg gathers 32.

Other fastening elements also can be supplied and attached to garment 10 either prior to or downstream from garment forming station 800. It is preferred that fastening elements are secured to garment 10 after garment forming station 800. Additional layers 20 (FIG. 2) may be placed on or in garment 10 before or after forming station 800, or be disposed between top sheet 14 and back sheet 12 by supplying the additional layer(s) to garment forming station 800. These additional layer(s) 20 also may be cut-and-placed on top sheet 14 and/or back sheet 12 using a cut-and-place apparatus 400.

It also is typical in the industry to include a waist elastic system comprising one or more waist elastic materials 5. Waist elastics 5 preferably are supplied upstream of garment forming station 800, and thus positioned between the top sheet 14 and back sheet 12. Waist elastics 5 may, however, be supplied downstream from garment forming station 800, and placed within a fold at laterally extending waist edges 204, 205 of an outer cover or cover sheet 234 to be positioned outside the back sheet 12, using techniques known in the art. The waist elastic elements 5 may be supplied as a layer of material, or as elastic elements positioned between two outer materials. In a similar fashion, tummy elastics 3 may be supplied prior to, or downstream from garment forming station 800.

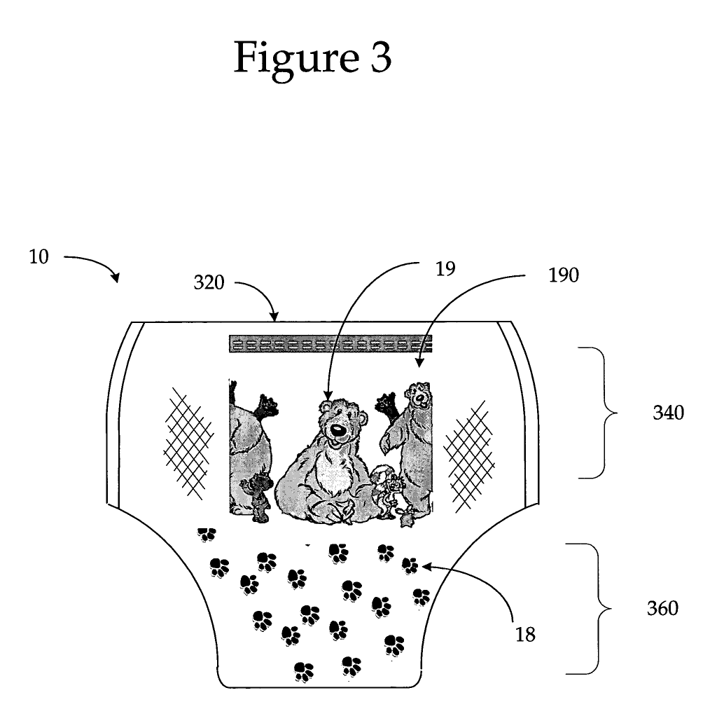

Turning now to FIGS. 3-5, various embodiments of the invention will be described with reference to particularly preferred embodiments. FIG. 3 illustrates an absorbent article 10, preferably a training pant, having a plurality of character graphics 19 positioned in the waist portion 340 that is adjacent a longitudinal edge 320 of the article 10. The article 10 also includes a plurality of wetness indicator graphics 18 that are positioned in the crotch portion 360 that is adjacent the waist portion 340. The character graphics 19 are printed on a web 190, and then cut and placed in the waist portion 340 using the techniques described herein. In this particularly preferred embodiment, no registration of the character graphics 19 is required. As shown in FIG. 3, the wetness indicator graphics 18, (the paw prints), are related to the character graphics 19, (the animals). Because the character graphics 19 are not depicted in partaking in any activity, the wetness indicator graphics 18 are not interactively interrelated with the character graphics 19, nor are they related to any activity in which the character graphics 19 are partaking.

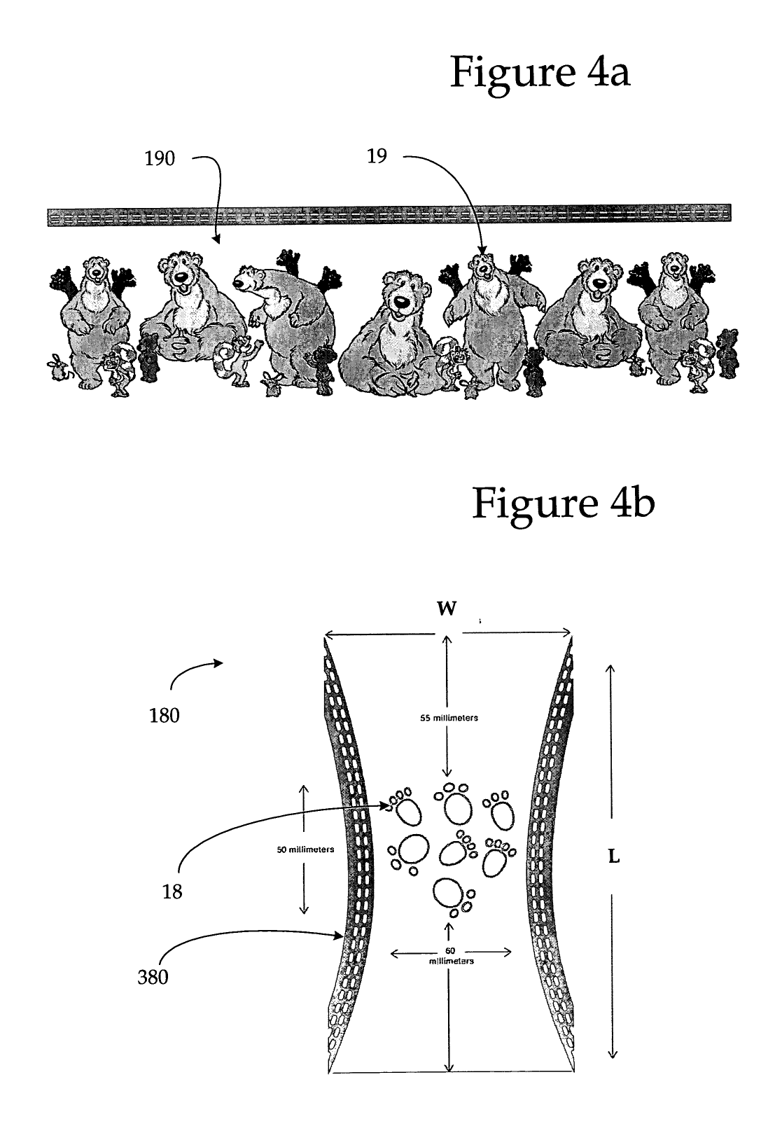

FIGS. 4a and 4b illustrate particularly preferred embodiments for printing character graphics 19 and wetness indicator graphics 18, without the need to register the particular graphics on the absorbent article. In FIG. 4a, a plurality of character graphics 19 are printed on a web 190. The character graphics 19 are printed in a repeating pattern along a continuous web 190, whereby only one pattern is shown in FIG. 4a. The group of character graphics 19 on the left-most side of the web 190 in FIG. 4a is the same as the group o f character graphics 19 on the right-most side of web 190. The character graphics 19 are grouped and sized in such a manner that they can be cut to a specific width to ensure that at least one of the main characters 19, (in this case a bear), is displayed fully in the resulting article after web 190 is cut and placed in the waist portion of the article. Those skilled in the art are capable of designing suitable character graphics 19, and positioning them in a repeating pattern on web 190 to ensure complete display of at least one character graphic 19 in the absorbent article 10, using the guidelines presented herein.

FIG. 4b illustrates a particularly preferred embodiment for printing wetness indicator graphics 18 on a web 180. As shown in FIG. 4b, a plurality of monochromatic bug silhouettes are shown as the wetness indicator graphics 18. These graphics can be printed in suitable disappearing ink, or in a suitable appearing ink. In addition, the graphics can be printed with an adhesive or other formulation that either disappears or appears upon the passage of time by being exposed to the atmosphere. These types of wetness indicators are known in the art and described, for example, in U.S. Pat. Nos. 6,297,424, and 6,307,119.

FIG. 4b also shows an additional graphic 380, that may be printed with a conventional ink, or with a wetness indicating substance. The additional graphic 380 displayed in FIG. 4b could be useful to reveal the sides of the leg openings of the absorbent article 10, when place on a user. In the embodiment shown, the entire web 180 can be positioned between the back sheet 12, and absorbent core 16 (FIG. 2), and preferably is sized such that its width (W) and length (L) are similar to the longitudinal and lateral dimensions of the crotch region of the garment. Thus, the web 180 can be attached to the back sheet 12 prior to feeding back sheet 12 to the garment forming station 800 (FIG. 6), or the graphics 18, 380, could be printed directly on the back sheet 12 material (preferably the absorbent core facing surface if a wetness indicator that appears or disappears upon being insulted with urine is used) during manufacture of the absorbent article 10.

The specific dimensions of the wetness indicator graphics 18, and their placement on web 180 are not critical to the invention. One preferred embodiment for a training pant are shown in FIG. 4b. As shown, the plurality of wetness indicator graphics 18 are about 60 mm in width, and 50 mm in length, each graphic 18 being about 15-20 mm in width and about 10-25 mm in length, or height. The wetness indicator graphics 18, when positioned in the crotch region of the article, preferably are printed together with additional graphic 380 in a pattern along the length of the web. The positioning of the pattern is such that when the web is cut and placed in the absorbent article, the pattern is positioned in the crotch region of the article. In one preferred embodiment when the entire web 180 is cut to a size similar in size to the absorbent article, the specific size of the cut web is dependent upon the size of the absorbent article, (e.g., stage of diaper or training pant), and preferably, the cut web 180 has a length of from about 300 to about 600 mm, more preferably from about 350 to about 500 mm, and most preferably from about 375 to about 450 mm. The width also can vary, and preferably is within the range of from about 100 to about 200 mm, more preferably from about 125 to about 175 mm, and most preferably about 155 mm.

As shown in one preferred embodiment in FIG. 4b, the wetness indicating graphics 18 preferably are positioned about 55 mm from a longitudinal edge of the pattern printed on web 180, and about 60 mm from another longitudinal edge of the pattern printed on web 180. The additional graphics 380 preferably are about 10 mm in thickness, and traverse the crotch region of the article. Particularly preferred lengths (L) of the pattern on web 180 can vary from about 100 to about 200 mm, more preferably from about 130 to about 180 mm, and most preferably about 165 mm. Particularly preferred widths (W) of the pattern printed on web 180 can vary from about 70 to about 200 mm, more preferably from about 90 to about 120 mm, and most preferably about 99-100 mm.

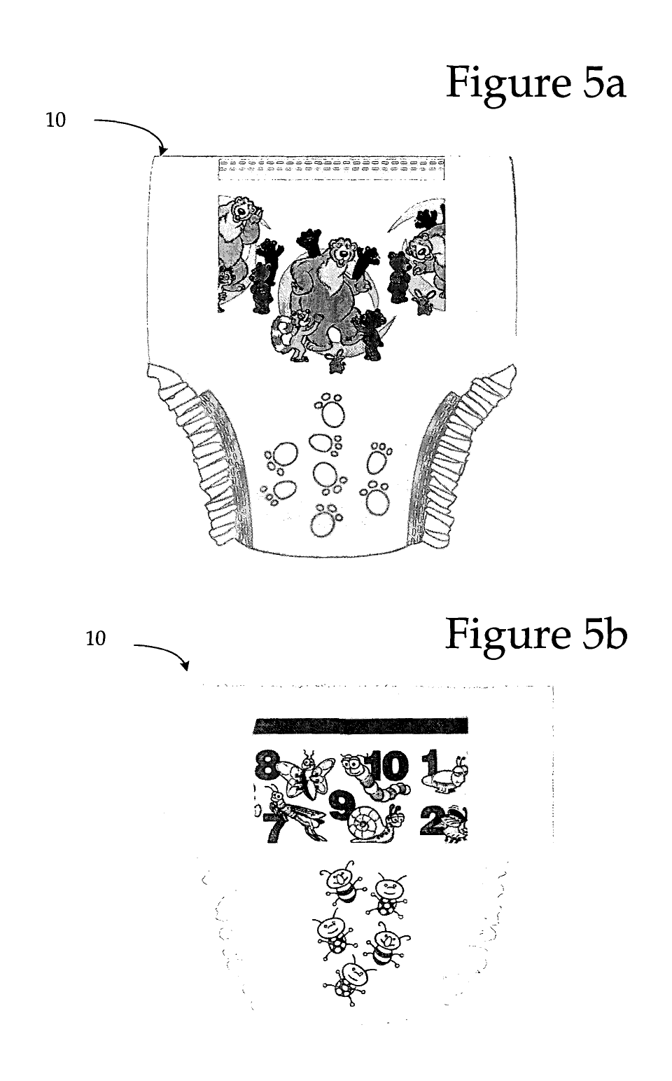

FIGS. 5a and 5b illustrate exemplary embodiments of the invention where the absorbent article 10 includes a plurality of character graphics in the waist portion and a plurality of wetness indicating graphics in the crotch region, whereby the wetness indicating graphics are related to the character graphics. FIG. 5a illustrates a plurality of animals from a well recognized children's television book series, the Bear in the Big Blue House®, and a plurality of related animal paw prints as wetness indicating graphics. FIG. 5b illustrates a plurality of bugs as character graphics, and a plurality of bug silhouettes as related wetness indicator graphics. In the most preferred embodiments shown in FIGS. 5a and 5b, the character graphics are not partaking in any activity, and if they are, the wetness indicators are related to the character graphics only, and not the character's particular activity.

The wetness indicator graphics 18 can be printed on any of the components of the absorbent article 10 using techniques known in the art. The graphics 18 can be printed directly on the back sheet 12, the absorbent core 16, or an intermediate web 180. Disappearing or fading graphics 18 preferably are printed with ink that disappears or fades when wetted, or when heated, or when exposed to the atmosphere for a period of time. The amount of fluid and/or heat and/or time needed to make the ink disappear should be less than the amount of fluid and/or heat generated during a normal insult of urine, which may depend upon the size of the absorbent article (and likewise the size of the wearer). Those skilled in the art are capable of using a suitable ink for any of the graphic elements described herein, using the guidelines provided in this disclosure.

Suitable inks or dyes useful in printing the wetness indicator graphics 18 of the present invention are those that disappear or fade when subjected to liquid and/or heat. Preferably, the ink(s) used to print wetness indicator graphics 18 is a water soluble ink of the type disclosed in U.S. Pat. No. 4,022,211. Examples of water soluble ink formulations include a water-soluble polyvinyl alcohol diluted with water, combined with appropriate coloring agents. For instance, a blue water soluble ink could be formulated from a 50% solution of Cascorex EA 9065, a polyvinyl alcohol from Borden Adhesives, diluted with distilled water with the addition of 0.1% by weight of GAF Neptune Blue BRA dye. Another suitable formulation is a solution comprised of 50% water, 50% of water-soluble polyvinyl alcohol (Cascorex EA 9065, about 8% solids from Borden Chemical Company), colored with a tissue dye (Sky Blue 6BX from E.I. DuPont Company) in the amount of 0.5% by weight. Coloring agents used in these formulations could be substituted with others, to produce different colors. Examples of other suitable coloring agents include: Pontamine Turquoise 8 GLP (a direct blue dye), Bond yellow CS (a direct yellow dye), DuPont Red 8BLX (a direct red dye), Rhodamine B Extra (a basic red dye), and Paper Blue R (a direct dye) all available from E.I. DuPont Company; and EASTACRYL dark red dye available from Eastman Kodak Company. Particularly preferred coloring agents include, for example, AquaDestruct inks bearing designations ESMSW5834784 (permanent blue), ESB507045SW (disappearing blue), ESMFW4834783 (permanent red), and ESMFW4834731 (disappearing red), all available from Sun Chemical Ink, Northlake, Ill. Coloring agents also could be added in different concentrations to produce different color intensities. Those skilled in the art are capable of designing and manufacturing a suitable ink for use in the invention, using the guidelines provided herein.

To the extent that the wetness indicator graphics 18 appear when contacted with urine and/or heat, suitable inks or dyes useful in printing the appearing graphics 18 are those that appear when subjected to liquid and/or heat. Preferably, the ink(s) used to print appearing graphics 18 is of the type disclosed in U.S. Pat. Nos. 4,292,916, 4,812,053, 4,903,254, 4,987,849, 5,045,283, 5,053,339, and 5,058,088, the disclosures of each of which are incorporated by reference herein in their entirety. Examples of suitable inks include ink formulations which are sensitive to pH (i.e. the pH of urine, when in contact with the ink, causes the printed graphic to appear.) Those skilled in the art are capable of designing and manufacturing a suitable appearing ink for use in the invention, using the guidelines provided herein.

The wetness indicator graphics 18 of various embodiments of the present invention also may be formed from appearing or disappearing adhesives that change colors or disappear, fade, or appear when wetted. The wetness indicator graphics 18 also may be printed with pH sensitive inks that disappear and/or appear when the surrounding pH changes due to, for example, an insult with urine. Any other type of ink or dye that is suitable in forming a disappearing or fading wetness indicator graphic 18 and/or an appearing wetness indicator graphic 18 can be used in the present invention.

To the extent the wetness indicator graphics 18 are of the type that appear or disappear over the passage of time due to contact with the air, the graphics 18 can be positioned anywhere on the article 10, and need not be positioned in the crotch portion. In particular, the wetness indicator graphic 18 can be responsive to time intervals, temperature levels, oxygen levels, or the like, and combinations thereof. Various visual indicators that appear over time in response to particular conditions are disclosed in U.S. Pat. No. 5,058,088; U.S. Pat. No. 5,053,339; U.S. Pat. No. 5,045,283; U.S. Pat. No. 4,987,849; U.S. Pat. No. 4,903,254; U.S. Pat. No. 4,812,053; and U.S. Pat. No. 4,292,916, all of which are incorporated herein by reference. A wetness indicator graphic 18 that appears or disappears over time may be applied to the product when use is initiated, or formed as an integral component of the product.

An additional embodiment of the invention encompasses one in which the character graphic(s) 19 is registered on the article in the waist region thereof. In this embodiment, the character graphic 19 may or may not be partaking in any activity, and the wetness indicator graphic 18 is related only to the character graphic 19, and not to the particular activity. For example, if the character graphic were an animal playing a musical instrument registered on the garment, the wetness indicator graphic could include a silhouette of that animal, a paw print of the animal, but would not include a graphic related to music.

An additional embodiment encompasses one in which the character graphics 19 represent anthropomorphic object or household items such as appliances (e.g., a toaster, an oven, a vacuum cleaner, etc.), and the wetness indicator graphics 18 are related to the character graphics. Related wetness indicator graphics 18 may include appliance cords and plugs, food, vacuum bags, and the like

The embodiments of the invention provide for an absorbent article that can be manufactured without complicated machinery and controls. The absorbent article also provides a toilet training tool to a caregiver by associating the wetness indicator graphics to the character graphics. In this instance, the caregiver and/or child being potty trained will know when an accident has occurred because the related wetness indicator (which has interested the child due to its relationship with the character graphic) has either appeared or disappeared.

While the invention has been described in detail with reference to particularly preferred embodiments and examples, those skilled in the art will appreciate that various modifications may be made to the invention without departing from the spirit and scope thereof.

1. An absorbent article comprising
a longitudinal end;
a waist portion positioned adjacent the longitudinal end; and
a crotch portion adjacent the waist portion,
wherein the absorbent article includes at least one unregistered character graphic positioned in the waist portion, and at least one wetness indicator graphic positioned in the crotch portion, and wherein the wetness indicator graphic and character graphic are related to one another, but not interactively interrelated.

2. The absorbent article as claimed in claim 1, further comprising a top sheet;
a back sheet; and
an absorbent core positioned at least partially between the top sheet and back sheet.

3. The absorbent article as claimed in claim 2, wherein the at least one wetness indicator graphic is positioned at least partially between the back sheet and the absorbent core.

4. The absorbent article as claimed in claim 2, wherein the at least one wetness indicator graphic is positioned on an absorbent core facing surface of the back sheet.

5. The absorbent article as claimed in claim 2, further comprising a web disposed at least partially between the absorbent core and the back sheet, and in fluid communication with the absorbent core, the web having printed thereon the at least one wetness indicator graphic.

6. The absorbent article of claim 1, further comprising a web of material positioned in the waist portion, wherein the unregistered character graphic is printed on the web of material.

7. The absorbent article of claim 6, wherein the web of material includes a repeating series of character graphics.

8. The absorbent article of claim 1, wherein the character graphic is selected from the group consisting of an animal, a cartoon character, and an anthropomorphic object and the wetness indicator graphic is a representative foot print of that character graphic.

9. The absorbent article of claim 8, wherein the animal is at least one bear, and the wetness indicator graphic is a paw print.

10. The absorbent article of claim 1, wherein the character graphic is an animal and the wetness indicator graphic is a silhouette of the animal.

11. The absorbent article of claim 10, wherein the animal is at least one bug.

12. The absorbent article of claim 1, wherein the wetness indicator graphic disappears or fades when insulted with urine.

13. The absorbent article of claim 1, wherein the wetness indicator graphic disappears or fades over a period of time when exposed to air.

14. The absorbent article as claimed in claim 1, wherein the article is selected from the group consisting of diapers, training pants, and adult incontinence products.

15. An absorbent article comprising:
a top sheet;
a back sheet;
an absorbent core positioned at least partially between the top sheet and the back sheet;
at least one wetness indicator graphic positioned on the absorbent core facing surface of the back sheet; and
at least one unregistered character graphic positioned in a waist portion of the absorbent article,
wherein the wetness indicator graphic and character graphic are related to one another, but not interactively interrelated.

16. The absorbent article as claimed in claim 15, further comprising a web disposed at least partially between the absorbent core and the back sheet, and in fluid communication with the absorbent core, the web having printed thereon the at least one wetness indicator graphic.

17. The absorbent article of claim 15, further comprising a web of material positioned in the waist portion, wherein the unregistered character graphic is printed on the web of material.

18. The absorbent article of claim 17, wherein the web of material includes a repeating series of character graphics.

19. The absorbent article of claim 15, wherein the character graphic is selected from the group consisting of an animal, a cartoon character, and an anthropomorphic object and the wetness indicator graphic is a representative foot print of that character graphic.

20. The absorbent article of claim 19, wherein the animal is at least one bear, and the wetness indicator graphic is a paw print.

21. The absorbent article of claim 15, wherein the character graphic is an animal and the wetness indicator is a silhouette of the animal.

22. The absorbent article of claim 21, wherein the animal is at least one bug.

23. The absorbent article of claim 15, wherein the wetness indicator graphic disappears or fades when insulted with urine.

24. The absorbent article of claim 15, wherein the wetness indicator graphic disappears over a period of time when exposed to the air.

25. The absorbent article as claimed in claim 15, wherein the article is selected from the group consisting of diapers, training pants, and adult incontinence products.

26. A method of making an absorbent article comprising:
providing a top sheet;
providing a back sheet;
providing an absorbent core and positioning the absorbent core at least partially between the top sheet and the back sheet;
positioning at least one wetness indicator graphic at least partially between the back sheet and the absorbent core; and
positioning at least one unregistered graphic in a waist portion of the absorbent article,
wherein the wetness indicator graphic and unregistered character graphic are related to one another, but not interactively interrelated.

27. The method as claimed in claim 26, further comprising providing a web having printed thereon the at least one wetness indicator graphic; and
positioning the web at least partially between the absorbent core and the back sheet such that the web is in fluid communication with the absorbent core.

28. The method as claimed in claim 26, further comprising providing a web having printed therein a repeating series of character graphics;
cutting the web at predetermined intervals; and
positioning the web in the waist portion of the absorbent article.

29. The method as claimed in claim 28, wherein cutting the web at predetermined intervals comprises cutting the web at a length greater than the length of at least one set of character graphics in the repeating series.

30. The method as claimed in claim 29, wherein cutting the web at predetermined intervals results in at least one full character graphic positioned in the waist portion.

31. The method as claimed in claim 26, wherein the wetness indicator graphic is printed directly on the back sheet.

32. The method of claim 26, wherein the character graphic is selected from the group consisting of an animal, a cartoon character, and an anthropomorphic object, and the wetness indicator graphic is a representative foot print of that character graphic.

33. The method of claim 32, wherein the animal is at least one bear, and the wetness indicator graphic is a paw print.

34. The method of claim 26, wherein the character graphic is an animal and the wetness indicator graphic is a silhouette of the animal.

35. The method of claim 34, wherein the animal is at least one bug.

36. The method of claim 26, wherein the wetness indicator graphic disappears or fades when insulted with urine.

37. The method of claim 26, wherein the wetness indicator graphic disappears or fades over a period of time when exposed to air.

38. The method of claim 26, wherein the article is selected from the group consisting of diapers, training pants, and adult incontinence products.

39. An absorbent article comprising:
a longitudinal end;
a waist portion positioned adjacent the longitudinal end;
a crotch portion adjacent the waist portion;
at least one character graphic positioned in the waist portion; and
at least one wetness indicator graphic positioned in the crotch portion,
wherein the wetness indicator graphic and character graphic are related to one another.

40. The absorbent article as claimed in claim 39, further comprising
a top sheet;
a back sheet; and
an absorbent core positioned at least partially between the top sheet and back sheet.

41. The absorbent article as claimed in claim 40, wherein the at least one wetness indicator graphic is positioned at least partially between the back sheet and the absorbent core.

42. The absorbent article as claimed in claim 40, wherein the at least one wetness indicator graphic is positioned on an absorbent core facing surface of the back sheet.

43. The absorbent article as claimed in claim 40, further comprising a web disposed at least partially between the absorbent core and the back sheet, and in fluid communication with the absorbent core, the web having printed thereon the at least one wetness indicator graphic.

44. The absorbent article of claim 39, further comprising a web of material positioned in the waist portion, wherein the character graphic is printed on the web of material.

45. The absorbent article of claim 44, wherein the web of material includes a repeating series of character graphics.

46. The absorbent article of claim 39, wherein the character graphic is selected from the group consisting of an animal, a cartoon character, and an anthropomorphic object, and the wetness indicator graphic is a representative foot print of that character graphic.

47. The absorbent article of claim 46, wherein the animal is at least one bear and the wetness indicator graphic is a paw print.

48. The absorbent article of claim 39, wherein the character graphic is an animal and the wetness indicator is a silhouette of the animal.

49. The absorbent article of claim 48, wherein the animal is at least one bug.

50. The absorbent article of claim 39, wherein the wetness indicator graphic disappears or fades when insulted with urine.

51. The absorbent article of claim 39, wherein the wetness indicator graphic disappears or fades over a period of time when exposed to the air.

52. The absorbent article of claim 39, wherein the article is selected from the group consisting of diapers, training pants, and adult incontinence products.

53. An absorbent article comprising:
a longitudinal end;
a waist portion positioned adjacent the longitudinal end;
a crotch portion adjacent the waist portion;
at least one anthropomorphic object graphic positioned in the waist portion; and
at least one wetness indicator graphic positioned in the crotch portion,
wherein the wetness indicator graphic and anthropomorphic object graphic are related to one another.

54. The absorbent article as claimed in claim 53, further comprising a top sheet;
a back sheet; and
an absorbent core positioned at least partially between the top sheet and back sheet.

55. The absorbent article as claimed in claim 54, wherein the at least one wetness indicator graphic is positioned at least partially between the back sheet and the absorbent core.

56. The absorbent article as claimed in claim 54, wherein the at least one wetness indicator graphic is positioned on an absorbent core facing surface of the back sheet.

57. The absorbent article as claimed in claim 54, further comprising a web disposed at least partially between the absorbent core and the back sheet, and in fluid communication with the absorbent core, the web having printed thereon the at least one wetness indicator graphic.

58. The absorbent article of claim 53, further comprising a web of material positioned in the waist portion, wherein the anthropomorphic object graphic is printed on the web of material.

59. The absorbent article of claim 58, wherein the web of material includes a repeating series of anthropomorphic object graphics.

60. The absorbent article of claim 53, wherein the anthropomorphic object graphic is an anthropomorphic household appliance, and the wetness indicator graphic is related to the household appliance.

61. The absorbent article of claim 60, wherein the anthropomorphic object graphic is at least one toaster and the wetness indicator graphic is a power plug.

62. The absorbent article of claim 53, wherein the wetness indicator is a silhouette of the anthropomorphic object graphic.

63. The absorbent article of claim 62, wherein the anthropomorphic object graphic is at least one toaster.

64. The absorbent article of claim 53, wherein the wetness indicator graphic disappears or fades when insulted with urine.

65. The absorbent article of claim 53, wherein the wetness indicator graphic disappears or fades over a period of time when exposed to the air.

66. The absorbent article of claim 53, wherein the article is selected from the group consisting of diapers, training pants, and adult incontinence products.
